(19) United States
(12) Patent Application Publication
(10) Pub. No.: US 2026/0056181 A1
(43) Pub. Date: Feb. 26, 2026

(54) Methods and System for Detecting Subsurface High-Pressure Gas Reservoirs

(71) Applicant: California Institute of Technology, Pasadena, CA (US)

(72) Inventor: Kenneth A. Farley, Altadena, CA (US)

(73) Assignee: California Institute of Technology, Pasadena, CA (US)

(21) Appl. No.: 19/308,082

(22) Filed: Aug. 22, 2025

Related U.S. Application Data
(60) Provisional application No. 63/686,277, filed on Aug. 23, 2024.

(51) Int. Cl. G01N 33/24; G01N 33/00

(52) U.S. Cl. CPC G01N 33/24; G01N 33/0067

(57) ABSTRACT

Methods and systems for detecting subsurface high-pressure helium or hydrogen reservoirs are described. Various methods and systems analyze helium or hydrogen concentrations in rocks to identify and determine the purity of helium or hydrogen in subsurface reservoirs.

### CROSS-REFERENCE TO RELATED APPLICATIONS

The current application claims the benefit, under 35 U.S.C. § 119(e), of U.S. Provisional Patent Application No. 63/686,277, entitled “Detection of Subsurface High-Pressure Helium Reservoirs” filed Aug. 23, 2024. The disclosure of U.S. Provisional Patent Application No. 63/686,277 is hereby incorporated by reference in its entirety for all purposes.

### FIELD OF THE INVENTION

The present disclosure generally relates to detection of subsurface gas reservoirs, in particular to detection of subsurface high-pressure helium and/or hydrogen reservoirs.

### BACKGROUND OF THE INVENTION

Helium is a finite resource that is being extracted from underground. Identification of high pressure and high purity subsurface helium is an important step in exploration for commercially viable helium reservoirs. Two methods are currently in use for helium exploration. In the first, soil gas is measured for helium concentration. If helium leaks from the subsurface reservoir into the soil, a detection can be made. This method is inadequately sensitive and not diagnostic regarding depth, purity, and pressure of any subsurface helium reservoir. The alternative is to drill a well (or occupy an existing well) and directly analyze well gas for helium concentration. This is a costly approach to use for exploration given permitting requirements and costs for well drilling. Using existing wells is not viable in the many promising exploration target regions where oil and gas wells are uncommon or nonexistent (e.g., igneous rocks).

Commercial hydrogen has to be manufactured, either in a polluting way, by using fossil fuels, or in an expensive way, by using renewable electricity. Natural hydrogen is molecular hydrogen present on Earth that is formed by natural processes such as radiolysis, serpentinization. Natural hydrogen can be trapped by rocks, forming subsurface reservoirs. The naturally occurring hydrogen can be potentially a clean energy source. However, similar to helium reservoirs, exploration methods for subsurface hydrogen reservoirs primarily rely on dry soil gas studies or gas vent measurements.

### SUMMARY OF THE INVENTION

Many embodiments are directed to methods and systems for detecting subsurface high-pressure gas reservoirs. Several embodiments detect subsurface high-pressure helium and/or hydrogen reservoirs by analyzing helium and/or hydrogen concentrations in nearby rocks. Some embodiments analyze isotopic compositions of helium in the rocks as a part of the detection processes.

Some embodiments include a method for detecting a subsurface helium reservoir, comprising: measuring helium concentration from at least one mineral isolated from a rock; determining a helium partial pressure based on the helium concentration in the at least one mineral; and determining a purity of helium in the subsurface helium reservoir based on the helium partial pressure.

In some embodiments, the rock is a core or cutting from a repository.

Some embodiments further comprise separating the at least one mineral from the rock.

Some embodiments further comprise extracting helium from the at least one mineral and purifying the extracted helium.

In some embodiments, extracting helium comprises a vacuum heating, fusion, or crushing process.

Some embodiments further comprise determining and calibrating helium solubility in the at least one mineral.

In some embodiments, the helium concentration in the at least one mineral is based on the helium solubility in the at least one mineral and the helium partial pressure.

In some embodiments, measuring helium concentration comprises measuring concentrations of helium-3 and helium-4 respectively from the at least one mineral.

In some embodiments, measuring concentrations of helium-3 and helium-4 using a mass spectrometer.

In some embodiments, the at least one mineral is nearly free of uranium and thorium.

In some embodiments, the at least one mineral is plagioclase and/or quartz.

In some embodiments, the helium partial pressure is based on a lithostatic pressure and the purity of helium in the subsurface helium reservoir.

Some embodiments include a method for detecting a subsurface gas reservoir, comprising: measuring a concentration of a gas from at least one mineral isolated from a rock; determining a partial pressure of the gas based on the gas concentration in the at least one mineral; and determining a purity of the gas in the subsurface gas reservoir based on the partial pressure.

In some embodiments, the gas is helium or hydrogen.

In some embodiments, the rock is a core or cutting from a repository.

Some embodiments further comprise separating the at least one mineral from the rock.

Some embodiments further comprise extracting the gas from the at least one mineral and purifying the extracted gas.

In some embodiments, extracting the gas comprises a vacuum heating, fusion or crushing process.

Some embodiments further comprise determining and calibrating a solubility of the gas in the at least one mineral.

In some embodiments, the gas concentration in the at least one mineral is based on the solubility in the at least one mineral and the partial pressure.

In some embodiments, measuring the gas concentration comprises measuring isotopic concentrations of the gas from the at least one mineral.

In some embodiments, measuring concentrations of the gas using a mass spectrometer or a gas chromatograph.

In some embodiments, the at least one mineral is nearly free of uranium and thorium.

In some embodiments, the at least one mineral is plagioclase and/or quartz.

In some embodiments, the partial pressure is based on a lithostatic pressure and the purity of the gas in the subsurface gas reservoir.

Additional embodiments and features are set forth in part in the description that follows, and in part will become apparent to those skilled in the art upon examination of the specification or may be learned by the practice of the disclosure. A further understanding of the nature and advantages of the present disclosure may be realized by reference to the remaining portions of the specification and the drawings, which forms a part of this disclosure.

### BRIEF DESCRIPTION OF THE DRAWINGS

The description will be more fully understood with reference to the following figures and data, which are presented as exemplary embodiments of the disclosure and should not be construed as a complete recitation of the scope of the invention, wherein:

FIG. 1 illustrates a schematic of detecting subsurface high-pressure helium reservoirs in accordance with an embodiment.

FIG. 2 illustrates a schematic of detecting subsurface high-pressure hydrogen reservoirs in accordance with an embodiment.

FIG. 3 illustrates measured helium concentrations from rock samples acquired at different depths in accordance with an embodiment.

### DETAILED DESCRIPTION OF THE INVENTION

A high pressure and high purity helium and/or hydrogen reservoir in the subsurface imprints its existence into the rocks that surround and overly the reservoir. Many embodiments provide methods for detecting subsurface helium reservoirs by analyzing the helium concentration and isotopic composition in certain mineral phases in rocks. Several embodiments provide methods for detecting subsurface hydrogen reservoirs by analyzing the hydrogen concentration in certain mineral phases in rocks.

When minerals are in contact with high pressure helium or hydrogen gas, helium or hydrogen dissolves into the minerals. Other naturally occurring gases (such as methane) and inert gases (such as neon, argon) are too big to be dissolved into minerals. Dissolved helium or hydrogen can be detected and quantified in accordance with many embodiments. The minerals store the dissolved helium or hydrogen. In other words, if a mineral sample is collected by drilling that occurs potentially years before the analysis, the identification processes in accordance with many embodiments can still accurately identify the dissolved helium or hydrogen and their concentrations.

Helium or hydrogen dissolution into a mineral is controlled by the partial pressure of the gas and the properties of the specific mineral. In the subsurface, the total pressure of a free gas phase is typically controlled by the height of the overlying column of rock, i.e. lithostatic pressure. Lithostatic pressure can be readily calculated from rock density and subsurface depth. The partial pressure of helium in a subsurface gas reservoir is the product of the total (lithostatic) pressure and the mole fraction of helium in the gas (i.e., the helium purity of the reservoir). The partial pressure of hydrogen in a subsurface gas reservoir is the product of the total (lithostatic) pressure and the mole fraction of hydrogen in the gas (i.e., the hydrogen purity of the reservoir). Combining these two considerations, the existence and purity of helium or hydrogen in a subsurface reservoir can be deduced from the amount of helium or hydrogen dissolved in a mineral in contact with that reservoir.

In addition to dissolving in minerals, helium or hydrogen also dissolves into grain boundaries, where it can migrate quickly (in a geologic sense) away from the reservoir and especially towards the Earth's surface. Grain boundary helium or hydrogen also dissolves into minerals and can be measured. The migration process creates a wide halo of elevated helium or hydrogen concentrations in minerals around the subsurface helium or hydrogen reservoir respectively. In other words, a sample may not need to be from directly in the subsurface reservoir for analyzing helium or hydrogen concentration. A sample close to the subsurface reservoir such as (but not limited to) within 0.5 km to 1 km should contain helium or hydrogen for detecting subsurface reservoirs.

Systems and methods for detecting subsurface high-pressure helium or hydrogen reservoirs in accordance with various embodiments of the invention are discussed further below.

### Detection of Subsurface High-Pressure Helium Reservoirs

Many embodiments provide processes for detecting a subsurface high pressure and high purity helium reservoir by analyzing rocks. Although the operations of some of the disclosed methods are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods, systems, and apparatuses can be used in conjunction with other systems, methods, and apparatuses.

Helium has two isotopes helium-3 (³He) and helium-4 (⁴He). Helium-4 is produced by radioactive decay of uranium and thorium, and uranium and thorium are common elements in rocks. Thus, the presence of helium-4 in rocks can be from dissolution of helium from nearby reservoirs but can also be from radioactive decay of uranium and thorium. Identifying helium-4 and analyzing its concentration in the rocks may not accurately predict the presence (or absence) of helium reservoirs. In order to determine helium reservoirs by analyzing rocks, the analysis processes in accordance with many embodiments can accurately determine helium reservoirs by identifying helium-3 and analyzing its concentrations in the rocks. In other words, even in minerals that lack other sources of helium (radioactive decay of uranium and thorium), the dissolved helium-3 can be detected and quantified in accordance with many embodiments.

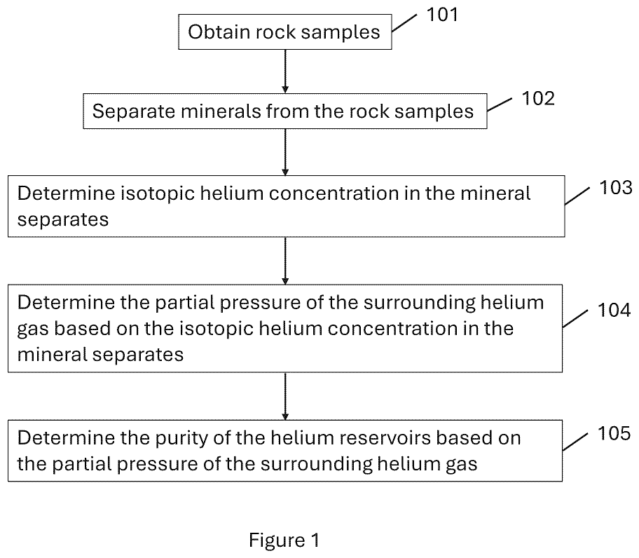

FIG. 1 illustrates a process for identifying subsurface high-pressure helium reservoirs in accordance with an embodiment of the invention. Several embodiments use readily available rock samples for identification 101. Examples of rock samples include (but are not limited to) cores and cuttings. In subsurface exploration, cores and cuttings are two types of rock samples extracted during drilling. Cores are cylindrical samples, typically obtained using a coring bit. Cuttings are smaller rock fragments brought to the surface by drilling fluid during rotary drilling. Cores and cuttings are frequently stored in a long-term repository for further study. The core and/or cutting samples in such a repository can be collected at various depths and at various times for purposes such as gold exploration, mineral exploration, oil exploration, etc. The rock samples can be from the area being targeted for helium exploration. Using readily available rock samples can largely reduce the cost for prospecting subsurface helium reservoirs.

Many embodiments separate the minerals from the rock samples 102. Mineral separation can isolate a mineral phase that can retain dissolved helium over periods of years between collection and analysis. Many minerals could meet this requirement such as (but not limited to) plagioclase and quartz. These phases are presumed to have high helium solubility at near-surface temperatures given their open crystal structure. In addition, these phases are presumed to equilibrate helium slowly with their surroundings—on a geologic timescale they equilibrate, but they will not equilibrate between sample collection and analysis in a laboratory.

Various methods can be used to separate the desired minerals from the rock samples. Certain embodiments use crushing and grinding to reduce the rock samples to smaller particles, releasing the individual mineral grains. Certain embodiments use visual sorting and/or sieving to separate the particles by size. Certain embodiments use microscopic examination to identify and separate the desired minerals. Certain embodiments use gravity separation (for example using liquids) to separate minerals based on their densities. Certain embodiments use magnetic separation to separate minerals based on their magnetic properties. In certain embodiments, the desired mineral grains can be selected under a microscope after mechanical disaggregation. As can readily be appreciated, any of a variety of separation methods can be utilized as appropriate to the requirements of specific applications in accordance with various embodiments of the invention.

After isolating an adequately pure mineral separate, many embodiments analyze the mineral separate for helium-3 and helium-4 concentrations 103. In several embodiments, analyzing helium-3 concentration rather than helium-4 gives a more accurate result for identification. Some embodiments use vacuum. Some mineral samples in a vacuum chamber to liberate the helium. The collected helium gas can be purified, for example by exposure to getters which consume reactive species, or by cryogenic techniques in which contaminants are removed by sorption at low temperature. Purified helium gas can be analyzed using a mass spectrometer such as (but not limited to) a helium isotope ratio sector-field mass spectrometer. The mass spectra identify and quantify helium-3 and helium-4 in the analyzed helium gas. As can readily be appreciated, any of a variety of methods can be utilized to measure helium-3 and helium-4 concentrations as appropriate to the requirements of specific applications in accordance with various embodiments of the invention.

The helium-3/helium-4 ratio of the separate can be used to help distinguish the helium source. The helium-3/helium-4 ratio, possibly supplemented by direct uranium, thorium, and lithium measurements, can be used to help distinguish pure radiogenic helium (helium from uranium and thorium decay, and possibly nuclear reactions of lithium) from helium derived from elsewhere and dissolved into the mineral either from a free helium gas phase or from grain boundaries. If the helium-3/helium-4 ratio is consistent with derivation of the helium by dissolution, the helium concentration can be used to infer the likelihood of a subsurface high pressure and high purity helium reservoir nearby.

Many embodiments determine the partial pressure of the surrounding helium using the helium concentration in the mineral separates 104. Helium dissolves into the minerals in proportion to the partial pressure of surrounding gaseous helium. In other words, when the helium gas is at a higher pressure, more helium dissolves into the minerals. When the helium gas is at a lower pressure, less helium dissolves into the minerals. According to Henry's law, helium concentration in the mineral equals to Henry's law constant times the partial pressure of helium in a subsurface gas reservoir. To determine the partial pressure of the surrounding helium, several embodiments perform calibration to establish helium solubility. In these calibrations test minerals are subjected to known pressures of helium, after which they are analyzed for helium concentration. These data can be used to determine the Henry's law (solubility) constant for the test mineral. The calibrations can be performed with each of the targeted minerals to determine helium solubility. Knowing the solubility of helium in the mineral and the experimentally measured helium concentration in the mineral, the partial pressure of subsurface helium can be calculated.

Many embodiments determine the purity of the helium reservoirs using the partial pressure of the surrounding helium 105. The partial pressure of subsurface helium reservoir is a product of the total lithostatic pressure and the mole fraction of helium in the gas. The total lithostatic pressure can be readily calculated, so one can derive helium purity in the subsurface reservoir.

While various processes for detecting subsurface high-pressure helium reservoirs are described above with reference to FIG. 1, any variety of processes that analyze isotopic helium concentrations in rocks to estimate the purity of the helium reservoirs can be utilized in identification and detection of subsurface helium reservoirs as appropriate to the requirements of specific applications in accordance with various embodiments of the invention.

### Detection of Subsurface High-Pressure Hydrogen Reservoirs

Many embodiments provide processes for detecting a subsurface high pressure and high purity hydrogen reservoir by analyzing rocks. Although the operations of some of the disclosed methods are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods, systems, and apparatuses can be used in conjunction with other systems, methods, and apparatuses.

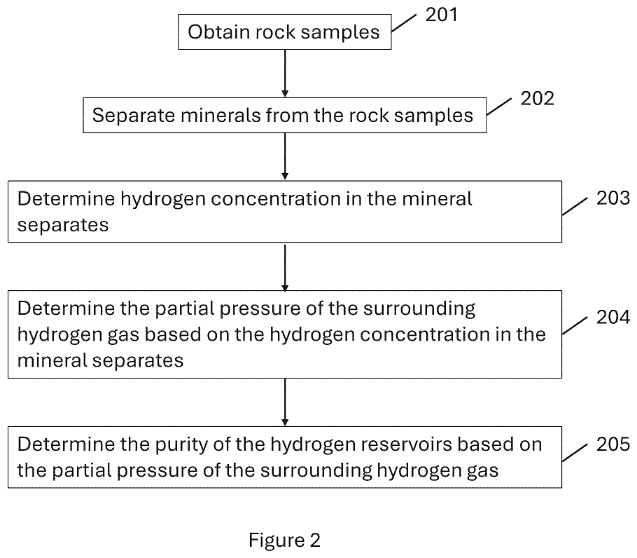

FIG. 2 illustrates a process for identifying subsurface high-pressure hydrogen reservoirs in accordance with an embodiment of the invention. Several embodiments use readily available rock samples for identification 201. Examples of rock samples include (but are not limited to) cores and cuttings. Cores and cuttings are stored in a publicly accessible repository. The core and/or cutting samples can be collected at various depths and at various times for purposes such as gold exploration, mineral exploration, oil exploration, etc. The rock samples can be from the area being targeted for hydrogen exploration. Using readily available rock samples can largely reduce the cost for prospecting subsurface hydrogen reservoirs.

Many embodiments separate the minerals from the rock samples 202. Mineral separation can isolate a mineral phase that is unlikely to have hydrogen implanted into it from nearby grains. Many minerals could meet this requirement such as (but not limited to) plagioclase and quartz. These phases are coarse grained (aiding separation) and are presumed to have high hydrogen solubility at near-surface temperatures given their open crystal structure. In addition, these phases are presumed to equilibrate hydrogen slowly with their surroundings—on a geologic timescale they equilibrate, but they will not equilibrate between sample collection and analysis in a laboratory.

Various methods can be used to separate the desired minerals from the rock samples. Certain embodiments use crushing and grinding to reduce the rock samples to smaller particles, releasing the individual mineral grains. Certain embodiments use visual sorting and/or sieving to separate the particles by size. Certain embodiments use microscopic examination to identify and separate the desired minerals. Certain embodiments use gravity separation (for example using liquids) to separate minerals based on their densities. Certain embodiments use magnetic separation to separate minerals based on their magnetic properties. In certain embodiments, the desired mineral grains can be selected under a microscope after mechanical disaggregation. As can readily be appreciated, any of a variety of separation methods can be utilized as appropriate to the requirements of specific applications in accordance with various embodiments of the invention.

After isolating an adequately pure mineral separate, many embodiments analyze the mineral separate for hydrogen concentrations 203. Some embodiments use vacuum crushing processes to collect hydrogen gas from the mineral separates. By this method the sample is pulverized in a vacuum chamber to release hydrogen in the mineral. If needed, the collected hydrogen gas can be purified of contaminants using cryogenic techniques. Liberated hydrogen gas can be analyzed using a mass spectrometer or by gas chromatography. The mass spectra or gas chromatogram identify and quantify hydrogen in the analyzed hydrogen gas. As can readily be appreciated, any of a variety of methods can be utilized to measure hydrogen concentrations as appropriate to the requirements of specific applications in accordance with various embodiments of the invention.

Many embodiments determine the partial pressure of the surrounding hydrogen using the hydrogen concentration in the mineral separates 204. hydrogen dissolves into the minerals in proportion to the partial pressure of surrounding gaseous hydrogen. In other words, when the hydrogen gas is at a higher pressure, more hydrogen dissolves into the minerals. When the hydrogen gas is at a lower pressure, less hydrogen dissolves into the minerals. According to Henry's law, hydrogen concentration in the mineral equals to Henry's law constant times the partial pressure of hydrogen in a subsurface gas reservoir. To determine the partial pressure of the surrounding hydrogen, several embodiments perform calibration to establish hydrogen solubility. The calibrations can be performed with each of the targeted minerals to determine hydrogen solubility. In these calibrations, test minerals are subjected to known pressures of hydrogen, after which they are analyzed for hydrogen concentration. These data can be used to determine the Henry's law (solubility) constant for the test mineral. Knowing the solubility of hydrogen in the mineral and the experimentally measured hydrogen concentration in the mineral, the partial pressure of subsurface hydrogen can be calculated.

Many embodiments determine the purity of the hydrogen reservoirs using the partial pressure of the surrounding hydrogen 205. The partial pressure of subsurface hydrogen reservoir is a product of the total lithostatic pressure and the mole fraction of hydrogen in the gas. The total lithostatic pressure can be readily calculated, so one can derive hydrogen purity in the subsurface reservoir.

While various processes for detecting subsurface high-pressure hydrogen reservoirs are described above with reference to FIG. 2, any variety of processes that analyze hydrogen concentrations in rocks to estimate the purity of the hydrogen reservoirs can be utilized in identification and detection of subsurface hydrogen reservoirs as appropriate to the requirements of specific applications in accordance with various embodiments of the invention.

### Exemplary Embodiments

Although specific embodiments of systems and apparatuses are discussed in the following sections, it will be understood that these embodiments are provided as exemplary and are not intended to be limiting.

### Example 1: Helium Concentration in Rocks

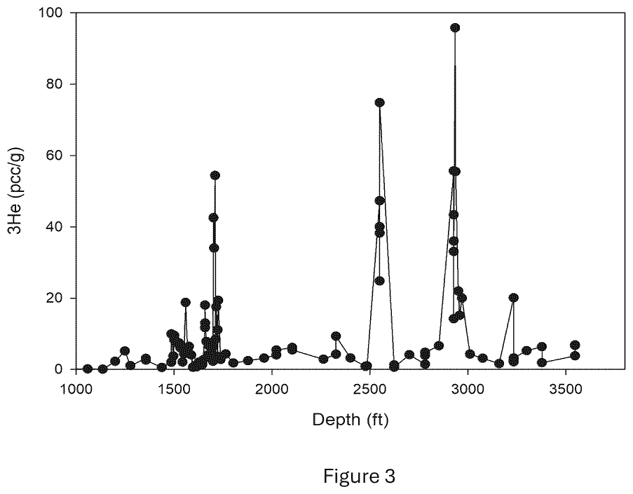

Many embodiments provide that helium concentrations can be accurately measured in the rock samples collected near a helium reservoir. FIG. 3 illustrates a schematic of helium concentration measured in samples from a rock core acquired in close proximity to a known high pressure helium reservoir in accordance with an embodiment. The known high pressure helium reservoir was discovered by analysis of gases coming from a well, at a depth of about 1650 feet. Helium concentration is measured with rock samples collected at various depths. The rock measurements, especially the peak at about 1700 feet, are consistent with the presence of this reservoir, and further suggest the existence of two deeper (about 2600 feet and about 2900 feet), and previously unknown, high pressure helium reservoirs.

### Doctrine of Equivalents

As can be inferred from the above discussion, the above-mentioned concepts can be implemented in a variety of arrangements in accordance with embodiments of the invention. Accordingly, although the present invention has been described in certain specific aspects, many additional modifications and variations would be apparent to those skilled in the art. It is therefore to be understood that the present invention may be practiced otherwise than specifically described. Thus, embodiments of the present invention should be considered in all respects as illustrative and not restrictive.

As used herein, the singular terms “a”, “an”, and “the” may include plural referents unless the context clearly dictates otherwise. Reference to an object in the singular is not intended to mean “one and only one” unless explicitly so stated, but rather “one or more”.

As used herein, the terms “approximately”, and “about” are used to describe and account for small variations. When used in conjunction with an event or circumstance, the terms can refer to instances in which the event or circumstance occurs precisely as well as instances in which the event or circumstance occurs to a close approximation. When used in conjunction with a numerical value, the terms can refer to a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to +0.1%, or less than or equal to ±0.05%.

Additionally, amounts, ratios, and other numerical values may sometimes be presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

What is claimed is:

1. A method for detecting a subsurface helium reservoir, comprising:
measuring helium concentration from at least one mineral isolated from a rock;
determining a helium partial pressure based on the helium concentration in the at least one mineral; and
determining a purity of helium in the subsurface helium reservoir based on the helium partial pressure.

2. The method of claim 1, wherein the rock is a core or cutting from a repository.

3. The method of claim 1, further comprising separating the at least one mineral from the rock.

4. The method of claim 1, further comprising extracting helium from the at least one mineral and purifying the extracted helium.

5. The method of claim 4, wherein extracting helium comprises a vacuum heating, fusion, or crushing process.

6. The method of claim 1, further comprising determining and calibrating helium solubility in the at least one mineral.

7. The method of claim 6, wherein the helium concentration in the at least one mineral is based on the helium solubility in the at least one mineral and the helium partial pressure.

8. The method of claim 1, wherein measuring helium concentration comprises measuring concentrations of helium-3 and helium-4 respectively from the at least one mineral.

9. The method of claim 8, wherein measuring concentrations of helium-3 and helium-4 using a mass spectrometer.

10. The method of claim 1, wherein the at least one mineral is nearly free of uranium and thorium.

11. The method of claim 1, wherein the at least one mineral is plagioclase and/or quartz.

12. The method of claim 1, wherein the helium partial pressure is based on a lithostatic pressure and the purity of helium in the subsurface helium reservoir.

13. A method for detecting a subsurface gas reservoir, comprising:
measuring a concentration of a gas from at least one mineral isolated from a rock;
determining a partial pressure of the gas based on the gas concentration in the at least one mineral; and
determining a purity of the gas in the subsurface gas reservoir based on the partial pressure.

14. The method of claim 13, wherein the gas is helium or hydrogen.

15. The method of claim 13 wherein the rock is a core or cutting from a repository.

16. The method of claim 13, further comprising separating the at least one mineral from the rock.

17. The method of claim 13, further comprising extracting the gas from the at least one mineral and purifying the extracted gas.

18. The method of claim 17, wherein extracting the gas comprises a vacuum heating, fusion or crushing process.

19. The method of claim 13, further comprising determining and calibrating a solubility of the gas in the at least one mineral.

20. The method of claim 19, wherein the gas concentration in the at least one mineral is based on the solubility in the at least one mineral and the partial pressure.

21. The method of claim 13, wherein measuring the gas concentration comprises measuring isotopic concentrations of the gas from the at least one mineral.

22. The method of claim 13, wherein measuring concentrations of the gas using a mass spectrometer or a gas chromatograph.

23. The method of claim 13, wherein the at least one mineral is nearly free of uranium and thorium.

24. The method of claim 13, wherein the at least one mineral is plagioclase and/or quartz.

25. The method of claim 13, wherein the partial pressure is based on a lithostatic pressure and the purity of the gas in the subsurface gas reservoir.
